(19) United States
(12) Patent Application Publication
(10) Pub. No.: US 2026/0136284 A1
(43) Pub. Date: May 14, 2026

(54) AMBIENT POWERED DEVICE AND MOBILE TELECOMMUNICATIONS NETWORK

(71) Applicant: VODAFONE GROUP SERVICES LIMITED, Newbury, Berkshire (GB)

(72) Inventor: Chandrika WORRALL, London (GB)

(21) Appl. No.: 19/120,045

(22) PCT Filed: Oct. 12, 2023
(86) PCT No.: PCT/GB2023/052644

§ 371 Date: Apr. 10, 2025

(30) Foreign Application Priority Data
Oct. 14, 2022 (GB) 2215224.3

(51) Int. Cl. H04W 52/02; G16H 40/67; H02J 50/20; H02J 50/40; H02J 50/80

(52) U.S. Cl. CPC H04W 52/0225; G16H 40/67; H02J 50/20; H02J 50/40; H02J 50/80

(57) ABSTRACT

A method of controlling an ambient-powered device over a mobile telecommunications network is provided. The method comprises receiving a signal indicating the ambient-powered device is to perform a desired operation. In response to receiving the signal, the method comprises transmitting, a first communication to initiate charging of the ambient-powered device, and transmitting, over the mobile telecommunications network, a second communication to cause the ambient-powered device to perform the desired operation. A method of operating an ambient-powered device with a mobile telecommunications network is also provided.

### FIELD OF THE DISCLOSURE

The present disclosure relates to the Internet of Things (IoT). In particular, the present disclosure relates to IoT devices which communicate with a mobile telecommunications network.

### BACKGROUND

The Internet of Things (IoT) relates to devices (IoT devices) with various sensing, processing, and communication technologies which are communicating with other devices and systems over a network, for example a mobile telecommunications network.

For example, IoT devices may include objects such as lights, heating systems, air conditioning systems, media systems, camera systems and the like. IoT devices differ from their conventional counterparts in that they include at least some capability to communicate with other devices over a network, for example a wireless network.

In order to provide network connected functionality, IoT devices need access to some form of power source. For many IoT devices, it may be possible to connect the IoT device to a mains power. Where access to mains power is not readily available, IoT devices may be provided with some form of battery to power the IoT device. In some applications where the IoT device is, for example, to be installed in an inaccessible location, the IoT device may be designed to have a very low power consumption, such that the battery provided with the IoT device can power the IoT device for an extended period of time (e.g. several months or even years). It will be appreciated that incorporating a battery into an IoT device, especially where the battery is intended to power the device for a number of years, adds cost, weight, and size to the IoT device.

Instead of being provided with a conventional battery, some IoT devices are provided with an alternative energy source. In particular, some battery-less IoT devices are configured to harvest energy from their surroundings. Typically, this involves harvesting energy from an radiative electromagnetic radiation source, for example a radiative electromagnetic radiation source provided by the network over which the IoT device communicates. Such IoT devices are effectively powered by their ambient surroundings and may be known as ambient-powered devices.

The present disclosure relates to improvements in, or at least commercially important alternatives to, a method controlling an ambient-powered device over a mobile telecommunications network and a method of operating an ambient-powered device.

### SUMMARY

According to a first aspect of the disclosure, a method of controlling an ambient-powered device over a mobile telecommunications network is provided. The method comprises:
receiving a signal indicating the ambient-powered device is to perform a desired operation;
in response to receiving the signal, transmitting, a first communication to initiate charging of the ambient-powered device; and
transmitting, over the mobile telecommunications network, a second communication to cause the ambient-powered device to perform the desired operation.

In general, it is understood that for a device (e.g. a mobile telephone) to be in constant communication with a mobile telecommunications network (i.e. constantly connected to the network) requires an ongoing exchange of messages between the mobile telephone and the mobile telecommunications network. Such an exchange requires bandwidth of the mobile telecommunications network, as well as a source of power for the device (e.g. a battery of the mobile telephone).

According to this disclosure, an ambient-powered device is understood to be a battery-less electronic device. As such, an ambient-powered device according to this disclosure obtains energy for its operation by harvesting energy from its surroundings. For example, an ambient-powered device may harvest energy from a radiative electromagnetic charging signal transmitted by a transmitter (or transmitters) associated with the ambient-powered device. Ambient-powered devices according to this disclosure may either harvest energy from its surroundings and use the harvested energy to immediately power the device. In some embodiments, the ambient-powered device may include a limited amount of energy storage, such as a capacitor. Thus, in some embodiments, the ambient-powered device can accumulate charge from e.g. a radiative electromagnetic charging signal for a period of time before they have accumulated sufficient charge to perform a desired operation. In general, it will be appreciated that ambient-powered devices may not be able to harvest sufficient energy from their ambient surroundings to be in constant operation, e.g. in constant contact with a mobile telecommunications network.

Thus, according to the method of the first aspect, the mobile telecommunications network may not be in constant contact with the ambient-powered device (i.e. the ambient-powered device may not be constantly connected to the mobile telecommunications network). Rather, on receiving the signal indicating that the ambient-powered device is to perform a desired operation, a first communication to initiate charging of the ambient-powered device may be transmitted. In some embodiments, the first communication may be a radiative electromagnetic charging signal transmitted by the mobile telecommunications network which charges the ambient-powered device. That is to say, in some embodiments, the ambient-powered electronic device may harvest energy from the first communication transmitted by the mobile telecommunications network. It will be appreciated that the first communication is only transmitted in response to the signal indicating that the ambient-powered device is to perform a desired operation. Thus, the mobile telecommunications network may preserve bandwidth by not attempting to constantly charge the ambient-powered device.

According to the first aspect, the mobile telecommunications network transmits a second communication to cause the ambient-powered device to perform the desired operation. According to the first aspect, the mobile telecommunication therefore only attempts to contact the ambient-powered device following an attempt to charge the device, thereby operating the network in a bandwidth-efficient manner.

By transmitting different communications to cause the ambient-powered device to charge (first communication) and to cause the ambient-powered device to perform the desired operation (second communication), the communication (i.e. the RF signal) the ambient-powered device uses to harvest energy from may be different (e.g. a different frequency) to the second communication transmitted by the mobile telecommunications network which is used to communicate with the ambient-powered device.

Preferably, the signal is received by the mobile telecommunications network. Preferably, in response to receiving the signal, the mobile telecommunications network transmits the first communication to initiate charging of the ambient-powered device. Thus, in some embodiments a mobile telecommunications network may be used to relay a signal demanding operation of an ambient-powered device to an ambient-powered device which results in the ambient-powered device performing the desired operation. As the mobile telecommunications network controls both the charging of the ambient-powered device and the instruction of the ambient-powered device to perform the desired operation, the mobile telecommunications network provides a single interface to the party (e.g. a control unit) sending the signal indicating the ambient-powered device is to perform a desired operation.

For example, in some embodiments, the method of the first aspect may be used to alert a user that a medical treatment is to be administered by the user. It has been realised that improved health outcomes can be delivered by providing fast and efficient ways to notify a ser of a medical treatment to be administered. For example, some medical conditions require rapid diagnoses and treatment in order to realise positive health outcomes. Accordingly, such situations may benefit from providing communications to assist a user to locate and identify an apparatus for administering a medical treatment. As such, an ambient-powered device according to this disclosure may be physically associated with the medical treatment, or with an apparatus for administering the medical treatment. In accordance with the first aspect, the ambient-powered device may be operated to assist a user to locate the associated medical treatment/medical treatment apparatus such that the user can administer the medical treatment in a timely manner.

Thus, in some embodiments, the mobile telecommunications network may receive a signal indicating a medical treatment is to be administered by a user. The signal may be transmitted from a control unit associated with the user. For example, the control unit may be monitoring a medical condition of the user, wherein upon determining that a medical treatment is to be administered by the user, the control unit transmits the signal to the mobile telecommunications network. The mobile telecommunications network may then cause the ambient-powered device to perform an operation associated with the medical treatment and the user. As such, the mobile telecommunications network may provide an interface between the control unit and the ambient-powered device.

The operation to be performed by the ambient-powered device may be to assist a user to locate the ambient-powered device and the associated medical treatment/medical treatment apparatus. For example, the ambient-powered device may output an alert to alert to enable a user to locate the associated medical treatment/medical treatment apparatus. Outputting an alert may comprise causing the ambient-powered device to make a sound (e.g. operate a speaker or buzzer of the ambient-powered device) or illuminate one or more lights of the ambient-powered device.

In some embodiments, after transmitting the first communication, the method further comprises waiting to receive a message from the ambient-powered device indicating the ambient-power device is charged before transmitting the second communication. For example, once sufficiently charged the ambient-powered device may transmit a communication to the mobile telecommunications network order to establish a connection with the mobile telecommunications network at which point the mobile telecommunications network transmit the second communication. In some embodiments, where the mobile telecommunications network does not receive a message from the ambient-powered device indicating the ambient-powered device is charged within a predetermined time period, the mobile telecommunications network may re-transmit the first communication to attempt to initiate a charging of the ambient-powered device again. The predetermined time period may correspond to an expected charging time of the ambient-powered device. For example, where the ambient-powered device 16 is expected to charge within 1 second of the transmission of the first communication, the predetermined time period may be about 2 seconds for example.

In some embodiments the first communication transmitted is a radiative electromagnetic charging signal which charges the ambient-powered device. That is to say, the mobile telecommunications network may transmit the first communication which comprises a radiative electromagnetic charging signal in order to directly charge the ambient-powered device. As such, the ambient-powered device harvests energy from the radiative electromagnetic charging signal.

In some embodiments, transmitting the first communication to initiate charging of the ambient-powered device comprises transmitting a first communication over the mobile telecommunications network to a local charging transmitter associated with the ambient-powered device. The first communication is configured to cause the local charging transmitter to transmit a radiative electromagnetic charging signal to the ambient-powered device. Accordingly, the mobile telecommunications network may act as an interface between a signal demanding operation of an ambient-powered device and a local charging transmitter which is capable of charging the ambient-powered device. By using the mobile telecommunications network to act as an interface between the source of the signal and the local charging transmitter, the local charging transmitter may be capable of interfacing with a wide range of different devices or applications. That is to say, the local charging device may interface with one or more internet-connected application via the mobile telecommunications network, or with one or more devices also connected to the mobile telecommunications network (or internet).

In some embodiments, the method further comprises receiving, over the mobile telecommunications network, location information of the ambient-powered device. In some embodiments, a plurality of local charging transmitters may be provided, wherein each local charging transmitter has an associated location. One of the local charging transmitters may be associated with the ambient-powered electronic device, based on location information of the ambient-powered device. That is to say, in some embodiments, the mobile telecommunications network may instruct one or more of the local charging transmitters to charge the ambient-powered device. Where location information is provided, one of the plurality of local charging transmitters may be associated with the ambient-powered device based on the location information of the ambient-powered device received by the mobile telecommunications network. Thus, the mobile telecommunications network may instruct the associated local charging transmitter to initiate charging of the ambient-powered device, but not the other non-associated local charging transmitters based on the location information. In some embodiments, the location information is received from a user terminal associated with the ambient-powered device. In some embodiments, a user terminal may comprise a mobile telephone, a smart phone, a tablet computer, or any other computing device which is in communication with the mobile telecommunications network. A user terminal may provide location information by way of a Global Position System (GPS) module or similar location tracking technology. In such cases, the location information of the user terminal may be used to infer a proximity of the ambient-powered device in order to provide an indication of which of the plurality of local charging transmitters which should be used to attempt to charge the ambient-powered device.

According to a second aspect of the disclosure, a method of operating an ambient-powered device with a mobile telecommunications network is provided. The method comprises receiving, by the ambient-powered device a radiative electromagnetic charging signal to cause the ambient-powered device to charge. The method also comprises receiving, by ambient-powered device, a communication from the mobile telecommunications network indicative of an operation to be performed by the ambient-powered device. In response to the communication, the ambient-powered device performs the operation. Thus, it will be appreciated that the ambient-powered device of the second aspect may be used in combination with the method of operating the mobile telecommunications network of the first aspect. Accordingly, the method of the second aspect may incorporate equivalent features of operating the ambient-powered device corresponding to those of the optional features of the first aspect and any associated advantages.

In some embodiments, the radioactive electromagnetic charging signal is received from the mobile telecommunications network or from a local charging transmitter.

In some embodiments, the operation to be performed comprises outputting and alert, preferably a sound and or a light. Thus, the ambient-powered device may comprise a speaker, buzzer, or one or more lights (e.g. Light Emitting Diodes) in order to output the alert.

In some embodiments, the radiative electromagnetic charging signal charges a capacitor of the ambient-powered device until the ambient-powered device has stored a pre-determined amount of charge, where in optionally upon storing the pre-determined amount of charge the ambient-powered device transmits a message to the mobile telecommunications network.

According to a third aspect of the disclosure, a mobile telecommunications network is provided. The mobile telecommunications network is configured to perform the method of the first aspect.

According to a fourth aspect of the disclosure, an ambient-powered device is provided. The ambient-powered device is configured to perform the method of the second aspect. It will be appreciated, that the ambient-powered device of the fourth aspect may be operated in conjunction with the mobile telecommunications network of the third aspect.

### BRIEF DESCRIPTION OF THE FIGURES

Embodiments of this disclosure will now be described with reference to the following figures in which:

FIG. 1 shows a block diagram of a system comprising a mobile telecommunications network and an ambient-powered device according to this disclosure;

FIG. 2 shows a block diagram of an ambient-powered device according to this disclosure;

FIG. 3 shows a block diagram of a method of controlling an ambient-powered device over a mobile telecommunications network according to this disclosure;

FIG. 4 shows a block diagram of a method of operating an ambient-powered device with a mobile telecommunications network;

FIG. 5 shows a block diagram of a system comprising a mobile telecommunications network, a local transmitter and an ambient-powered device according to this disclosure;

FIG. 6 shows another block diagram of a system comprising a mobile telecommunications network, a local transmitter, and an ambient-powered device according to this disclosure; and

FIG. 7 shows a further block diagram of a system comprising a mobile telecommunications network, a local transmitter, and an ambient-powered device according to this disclosure.

### DETAILED DESCRIPTION

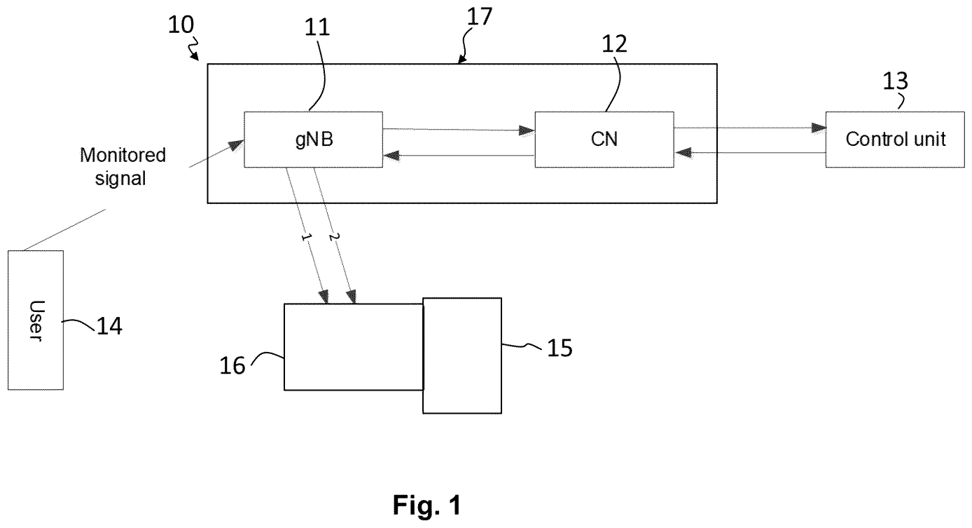

According to a first embodiment of the disclosure, a system 10 is provided. FIG. 1 shows a block diagram of the system 10 according to the first embodiment. The system 10 as shown in FIG. 1 is provided for notifying a user of a medical treatment to be administered. The system 10 comprises a user device 14, a mobile telecommunications network 17, a control unit 13, and an ambient-powered device 16. The ambient-powered device 16 is associated with an apparatus 15 for administering a medical treatment (or associated with a medical treatment). For example, in the embodiment of FIG. 1, the ambient-powered device 16 is physically connected to the apparatus 15 for administering a medical treatment.

The apparatus 15 for administering a medical treatment may, for example comprise a receptacle suitable for containing a medicament. In such examples, the medical treatment to be administered may comprise administering the medicament contained in the receptacle. In other examples, the apparatus 15 for administering the medical treatment may comprise a medical device for administering a treatment. In such examples, the medical treatment to be administered may comprise a treatment using the medical device.

The user device 14 may be any suitable device such as a mobile telephone (e.g. a smart phone), a tablet, a personal computer, a wearable device (e.g. a smart watch or other form of wearable electronic device) and the like. The user terminal 14 may be registered with the mobile telecommunications network 17 and may be capable of exchanging communications over the mobile telecommunications network 17 over an air interface. The user terminal 14 may be registered with the mobile telecommunications network and to a particular user of the user terminal 14.

In the embodiment of FIG. 1, the user terminal 14 may be a wearable device which is configured to monitor one or more conditions of the user. For example the monitored condition(s) may comprise one or more of: user movement, heart rate, body temperature, blood pressure, and blood glucose level. The user terminal 14 may communication the one or more monitored conditions to a control unit 13 via the mobile telecommunications network 17.

The mobile telecommunications network 17 includes a radio access network (RAN) and a core network (CN) 12. The RAN typically comprises a plurality of base stations 11 serving at least one cell. For ease of illustration, a single base station 11 is depicted in FIG. 1. In the embodiment of FIG. 1, the base station 11 is a Next Generation Node B (gNB) suitable for use as part of a 5G New Radio (NR) network. The base station 11 comprises at least one antenna configured to exchange communications (e.g. radio frequency signals with devices situated within a geographical coverage area) (which may be referred to as a cell serviced by the base station 11 over an air interface).

The base station 11 may exchange communications by transmitting and/or receiving communications in one or more frequency bands assigned to a radio access technology RAT) used by the base station 11 and utilising communication and protocols specified for the RAT (e.g. standardised communication protocols for the RAT). Suitable RAT may include, for example, the global system for mobile communication (GMS), the universal mobile telecommunications system (UMTS), long term evolution (LTE) and/or 5G new radio (NR). The base station 11 may take any suitable form and may, for example, comprise a GSM and/or UMTS compatible base station such as a nodeB, and evolved nodeB (eNB) and/or a 5G NRgnodeB. The base station 11 is in communication with the core network 12 via a suitable connection. The core network 12 provides network services to devices which are connected to the mobile telecommunications network 17 over the RAN. For example, the core network 12 may enable the user terminal 14 to be connected to the internet via the mobile telecommunications network 17.

The control unit 13 comprises one or more electronic devices configured to determine whether an operation is to be performed by the ambient-powered device 16. In the embodiment of FIG. 1 the control unit 13 is configured to determine a medical treatment to be administered by a user. The control unit 13 may take any suitable form such as one or more computing devices which may include one or more of a server, personal computer, tablet or smart phone and the like. The control unit 13 is capable of exchanging communications with the user terminal 14 via the mobile telecommunications network 17. For example, the control unit 13 may be connected to the interment and may receive communications from the user terminal 14 via its internet connection (which may be provide by the mobile telecommunications network 17). Additionally or alternatively, the control unit 14 may send communications to one or more devices connected to the mobile telecommunications network 17 via its internet connection. In embodiments where the user terminal 14 monitors one or more conditions of the user, the monitored conditions may be transmitted to the control unit 14. The control unit 14 may determine that a medical treatment is to be administered by a user based on the one or more monitored conditions from the user terminal 14. Upon making such a determination, the control unit may send a signal to the mobile telecommunications network 17 indicating the ambient-powered device 16 associated with the apparatus 15 is to perform a desired operation (output an alert).

In the embodiment of FIG. 1, the control unit 13 is shown as being separate to the mobile telecommunications network 17. In other embodiments, the control unit 13 may form part of the mobile telecommunications network 17. For example, the control unit 13 may form part of, or be in direct communication with, the core network 12 and/or the base station 11. In some embodiments, the control unit 13 may utilise any suitable connection arrangement for communication with the user terminal 14 and the ambient-powered device 16 over the mobile telecommunications network 17.

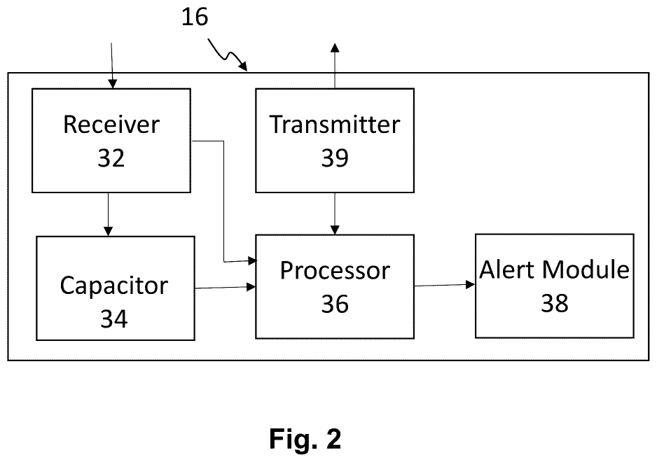

The ambient-powered device 16 is an electronic device configured to harvest energy from the environment. FIG. 2 shows a block diagram of an ambient-powered device according to this disclosure. The ambient-powered device of FIG. 2 comprises a receiver 32, a capacitor 34, a processor 36, an alert module, and a transmitter 39.

The ambient-powered device 16 is a battery-less device being instead powered by harvesting from energy sources characterized by lowest lower bounds of power density among the commonly known energy sources, for example radio frequency signals. The ambient-powered device 16 may be optimized for specific needs of services and applications requiring typically minimalistic static data read-out. As such, the maximum instantaneous communication power consumption of the ambient powered device may be no larger than tens of μW to a few hundred μW, depending on the operational requirements of the ambient-powered device.

The ambient-powered device of FIG. 2 is configured to harvest energy from a radiative electromagnetic charging signal. Such radiative electromagnetic charging signals may comprise radio frequency waves which propagate at the location of the ambient-powered device 16. Such electromagnetic waves may originate from a variety of different sources and may, for example, comprise signals transmitted over a mobile telecommunications network 17, wireless fidelity (Wi-Fi) signals, Bluetooth (RTM) signals and the like. Typically, the ambient-powered device 16 does not include an in-situ power source. For example, the ambient-powered device 16 of FIG. 2 does not include a battery and may not be connected (e.g. through a wired or wireless connection) to mains electricity. The ambient-powered device 16 may include a limited energy storage capability, for example a capacitor 34. Such limited energy storage capability may be capable of storing a small amount of energy suitable for performing a limited operation of the ambient-powered device 16. It will be appreciated that such limited energy storage capability is not capable of operating the ambient-powered device for an extended period of time.

The capacitor 34 may be selected in order to provide sufficient energy storage to allow the ambient-powered device 16 to perform the desired operation(s) once suitably charged. For example, in order to provide the ambient-powered device 16 with a power of about 100 μW for about 60 seconds would require a capacitor having a capacitance of around 3.2 mF.

The ambient-powered device also comprises a processor 36 which is configured to perform computational tasks. For example, the processor 36 may be configured to process communications received from the mobile telecommunications network 17 via receiver 32. The processor 36 may also communicate with the mobile telecommunications network 17 via transmitter 39 of the ambient-powered device 16. In some embodiments, transmitter 39 and receiver 32 may be provided as a transceiver.

The processor 36 of the ambient-powered device 16 may be configured to perform an operation on receipt of a second communication from the mobile telecommunications network instructing the ambient-powered device 16 to perform the operation. In the embodiment of FIG. 1, the ambient-powered device 16 may be configured to output an alert using alert module 38. The alert module 38 may comprise one or more of a speaker, a buzzer, or one or more lights (e.g. Light Emitting Diodes).

In other embodiments, the ambient-powered device 16 may be configured to perform other operations. For example, in some embodiments, the ambient-powered device 16 may comprise one or more sensor modules, said sensor module(s) configured to take a reading from a sensor. The ambient-powered device 16 may then transmit the sensor reading via the mobile telecommunications network to a server or control unit for example. As such, it will be appreciated that the desired operation to be performed by the ambient-powered device may include taking one or sensor readings, sounding an alert, and the like.

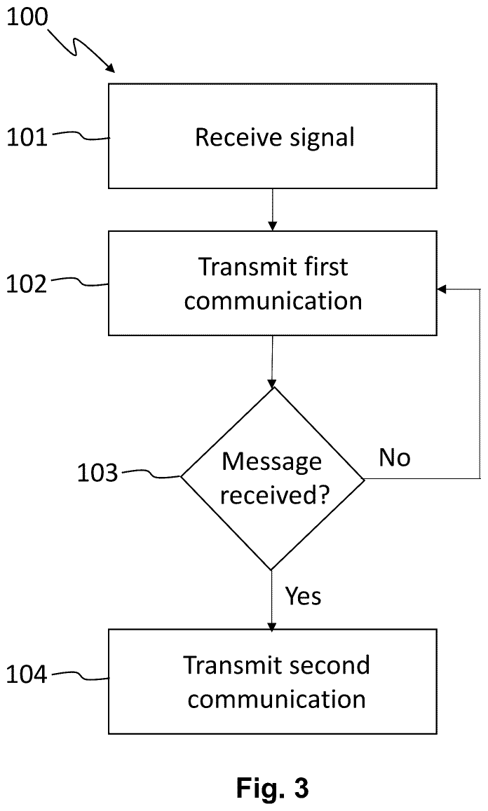

Next, a method 100 of operating a mobile telecommunication network of the system 10 shown in FIG. 1 will be described with reference to the block diagram of FIG. 3.

As shown in step 101 of the method 100, the mobile telecommunications network 17 receives a signal indicating the ambient-powered device is to perform a desired operation. In the embodiment of FIG. 1, the mobile telecommunications network receives the signal from control unit 13. In the embodiment of FIG. 1, the control unit 13 monitors a user. When control unit 13 determines that a medical treatment is to be administered by the user, the control unit 13 sends a signal to the mobile telecommunications network 17 to cause the ambient powered device 16 associated with the apparatus 15 to output an alert for the user.

In response to receiving the signal, in step 102 the mobile telecommunications network 17 transmits a first communication to initiate charging of the ambient-powered device 16. It will be appreciated that due to the limited energy storage capabilities of the ambient-powered device 16, the ambient-powered device 16 is not constantly powered. As such, the ambient-powered device 16 is not in constant contact with the mobile telecommunications network 17. Accordingly, in order to operate the ambient-powered device 16, the ambient-powered device 16 must first be charged. In the embodiment of FIG. 1, the first communication transmitted by the base station 11 is a radiative electromagnetic charging signal which charges the ambient-powered device 16.

The ambient-powered device 16 may receive the radiative electromagnetic charging signal and harvest the energy within the signal to charge a capacitor 34. As an example, the ambient-powered device 16 may be configured to harvest energy from a radiative electromagnetic charging signal a rate of at least 0.1 μW, preferably at least 1 μW, more preferably at least 10 μW. For example, the ambient-powered device 16 may be configured to harvest energy from a radiative electromagnetic charging signal a rate of about 10 μW, 20 μW, or 30 μW. Higher charging rates may particularly be achieved where a local charging transmitter is provided (see details below) ..

Once the ambient-powered device 16 is sufficiently charged, the ambient-powered device 16 may attempt to make contact with the mobile telecommunications network 17. As such, the ambient-powered device 16 may transmit a message to the mobile telecommunications network 17 to indicate that the charging of the ambient-powered device 16 was successful. The message may also prompt the mobile telecommunications network 17 to issue further instructions to the ambient-powered device 16.

As shown in FIG. 3, after transmitting the first communication, in step 103 the mobile telecommunications network 17 waits to receive the message from the ambient-powered device 16 indicating the ambient-powered device 16 has been charged. If the message is not received after a predetermined time period, the mobile telecommunications network may re-transmit the first communication (i.e. repeat step 102). The predetermined time period may be based on an expected charging time for the ambient-powered device 16. For example, where the ambient-powered device 16 is expected to charge in about 1 second, the mobile telecommunications network may wait about 2 seconds before re-transmitting the first communication.

In some embodiments, the mobile telecommunications network may repeat steps 102 and 103 to try to successfully charge the ambient-powered device 16 a predetermined number of times. For example, steps 102 and 103 may be repeated at least: 3, 5 7 or 10 times for example. In the event that the mobile telecommunications network 17 does not receive the expected message (e.g. the ambient-powered device 16 cannot be charged or is not operational), the mobile telecommunications network 17 may send a warning message to the control unit 13 or the user terminal 14 indicating that the ambient-powered device 16 could not be instructed to perform the operation. Thus, the mobile telecommunications network 17 may alert a user to any interruptions in the operation of the ambient-powered device 16.

When the mobile telecommunications network 17 does receive a message from the ambient-powered device 16, according to step 104, the mobile telecommunications network proceeds to transmit a second communication to the ambient-powered device 16. The second communication causes the ambient-powered device to perform the desired operation instructed by the control unit 13. In the embodiment of FIG. 1, the desired operation is for the ambient-powered device 16 to output an alert using the alert module 38 of the ambient-powered device 16.

On receipt of the second communication, the ambient-powered device 16 outputs the alert. The second communication transmitted by the mobile telecommunications network may include information describing the type of alert to be output. For example, the second communication may specify a duration of the alert to be output.

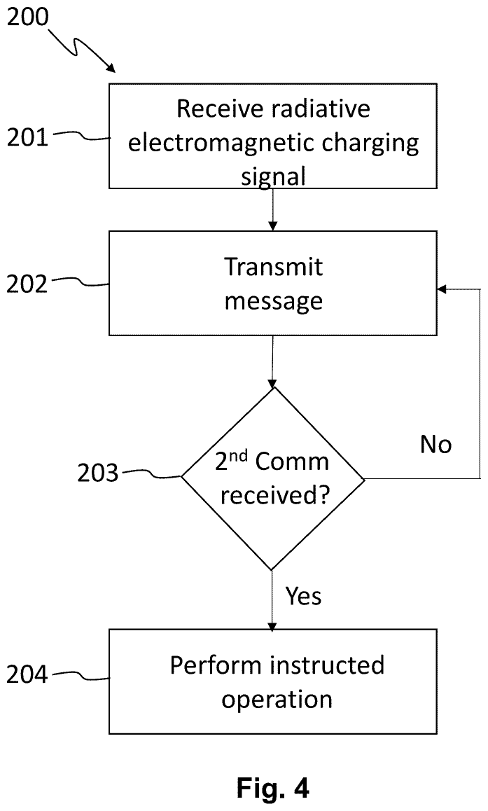

It will be appreciated that method 100 describes the steps performed by the mobile telecommunications network 17. FIG. 4 shows a block diagram of a method 200 of operating an ambient-powered device 16 with a mobile telecommunications network 17. The ambient-powered device 16 and mobile telecommunications network 17 may be provided as part of system 10 shown in FIG. 1.

As will be appreciated from the above description, the ambient-powered device 16 does not have a battery, or access to another power source to allow it to be in operation constantly. Thus, prior to step 201, the ambient-powered device 16 may be unpowered. As shown in step 201 of method 200 the ambient-powered device the receives a radiative electromagnetic charging signal from the mobile telecommunications network 17. The radiative electromagnetic charging signal charges the ambient-powered device 16. In the embodiment of FIG. 1, the radiative electromagnetic charging signal charges a capacitor 34 of the ambient-powered device 16 until the ambient-powered device has stored a predetermined amount of charge. The predetermined amount of charge may be detected by the ambient-powered device 16 when a voltage across the capacitor 34 reaches a predetermined level.

Upon storing the predetermined amount of charge, the ambient-powered device 16 transmits a message to the mobile telecommunications network (step 202 of method 200).

In some embodiments, the ambient-powered device 16 may then wait to receive further instructions from the mobile telecommunications network 17 (i.e. the second communication discussed above). In some embodiments, the ambient-powered device 16 may be configured to re-transmit the message (i.e. repeat step 202) if the second communication is not received from the mobile telecommunications network 17 within a predetermined time period. For example, the ambient-powered device 16 may wait about 10 seconds before repeating step 202 if the second communication is not received. It will be appreciated that the ambient-powered device 16 only has a limited amount of power. Thus, the ambient-powered device 16 may only have sufficient power to repeat step 202 one or two times.

As discussed above, the ambient-powered device 16 receives a communication from the mobile telecommunications network 17 indicative of an operation to be performed by the ambient-powered device 16. Upon receipt of the communication, in step 204 the ambient-powered device 16 performs the operation. In the embodiment of FIG. 1, the operation to be performed by the ambient-powered device 16 is to output an alert as discussed above. The methods 100 and 200 have been described above with reference to the system 10 shown in FIG. 1. It will be appreciated that the methods 100 and 200 may also be applied to different system architectures as discussed further below.

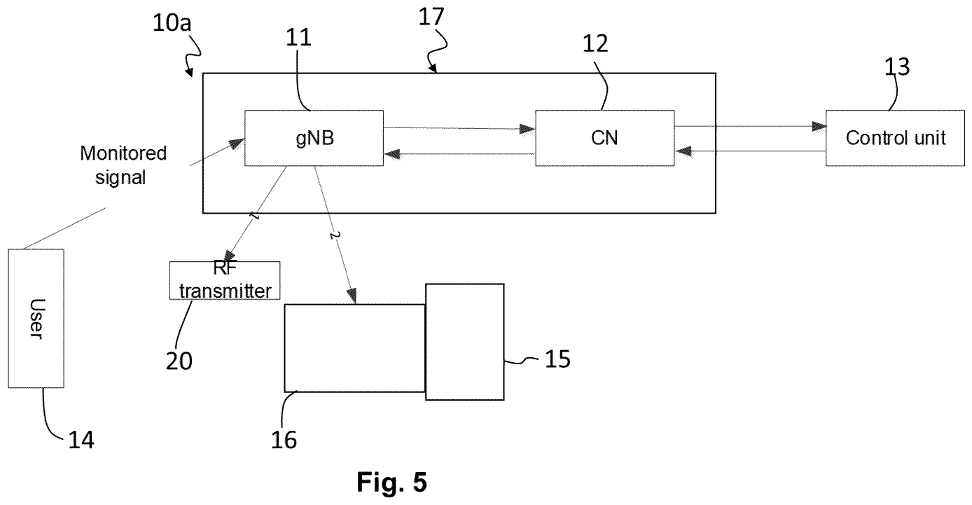

In some embodiments, the mobile telecommunications network 17 may not directly charge the ambient-powered device 16. Rather, a local charging transmitter 20 may be provided which is associated with the ambient-powered device 16. That is to say, a user may have a local charging transmitter 20 which is configured to transmit a radiative electromagnetic charging signal to charge the ambient-powered device 16. For example, for applications where the ambient-powered device 16 is expected to be located within a predetermined region (for example a user's home), a local charging device 20 may be used to transmit the radiative electromagnetic charging signal. By using a local charging device 20, the local charging device may be able to transmit a radiative charging signal of suitable intensity the ambient-powered device 16. An example of a system 10a comprising a mobile telecommunications network 17, an ambient-powered device 16 and a local charging transmitter 20 (RF transmitter) is shown in FIG. 5. It will be appreciated that there are various similarities between the components of the system 10 described above and the system 10a shown in FIG. 5. Where similar features are provided, the same reference numerals are used.

As shown in FIG. 5, the local charging transmitter 20 may communicate with the mobile telecommunications network 17 in order to receive the first communication indicating that the ambient-powered device 16 is to be charged. The first communication is configured to cause the local charging transmitter 20 to transmit a radiative electromagnetic charging signal to the ambient-powered device 16.

The radiative electromagnetic charging signal transmitted by the local charging transmitter to the ambient-powered device 16 may be any suitable signal to cause the ambient-powered device to charge. For example, in some embodiments, the ambient-powered device 16 is configured to harvest energy from RF frequencies within a predetermined range. Accordingly, the local charging transmitter 20 may transmit a radiative electromagnetic charging signal having a corresponding frequency in order to improve the efficiency of the energy harvesting. For example, in some embodiments the local charging transmitter may transmit a radiative electromagnetic charging signal in the RF frequency band having a frequency of at least 3 kHz and no greater than about 300 GHz.

In some embodiments, the mobile telecommunications network 17 may be configured to communicate with a plurality of local charging transmitters 20. Each local charging transmitter 20 may be associated with one or more ambient-powered devices 16. An ambient-powered device 16 may be associated with one or more local charging transmitters. Thus, in some embodiments, the mobile telecommunications network 17 may instruct only the local charging transmitters 20 associated with the ambient-powered device 16 to be operated to output a radiative electromagnetic charging signal. Thus, the system 10a may selectively charge only ambient-powered devices 16 which are intended to be operated.

In other embodiments, the mobile telecommunications network 17 may send a first communication to one local charging transmitter 20 of a plurality of local charging transmitters based on location information of the ambient-powered device 16. For example location information of the ambient-powered device 16 may be received by the mobile telecommunications network 17 from the control unit 13 or from a user terminal 14 associated with the user of the ambient-powered device 16. In some embodiments, location information of the user terminal 14 or location information of the control unit 13 may be indicative of the location of the ambient-powered device 16. As such, the mobile telecommunications network may send the first communication to a local charging device 20 which is located closest to the location indicated by the location information.

Once the local charging transmitter 20 has charged the ambient-powered device 16, the ambient powered device 16 may send a message to the mobile telecommunications network 17 as described above. Alternatively, the mobile telecommunications network may wait a period of time after sending the first communication before sending the second communication to the ambient-powered device 16.

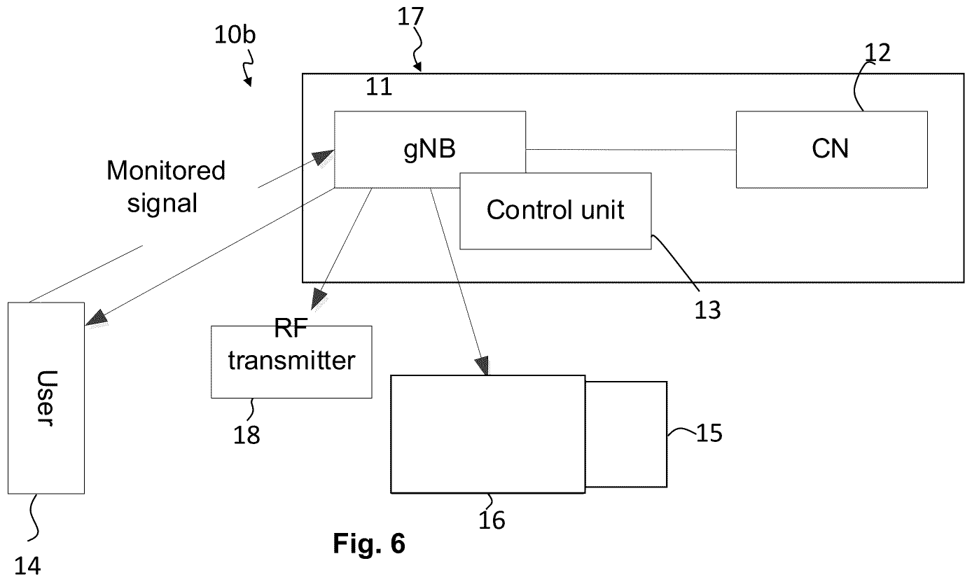

In some embodiments described above, the control unit 13 is a separate device to the mobile telecommunication network 17. In other embodiments, the control unit 13 may be provided as part of the mobile telecommunications network 17. FIG. 6 shows a block diagram of a system 10b where the control unit 13 is provided as part of the mobile telecommunications network 17. For example, the control unit 13 may form part of, or be in direct communication with, the core network 12 and/or the base station 11. For example, in some embodiment, the control 13 may located at the base station (gNB). This provides the possibility to have local control of the ambient-powered device 16 which may provide faster responses. Such an application may be particularly applicable to the embodiments of the disclosure in a medical, or care home setting.

As will be appreciated from the embodiment of FIG. 6, the mobile telecommunications network 17 may receive a signal from a control unit 13 forming part of the mobile telecommunications network indicating that the ambient-powered device 16 is to perform a desired operation. The mobile telecommunications network may the proceed to control the ambient-powered device 16 in line with method 100 as described above.

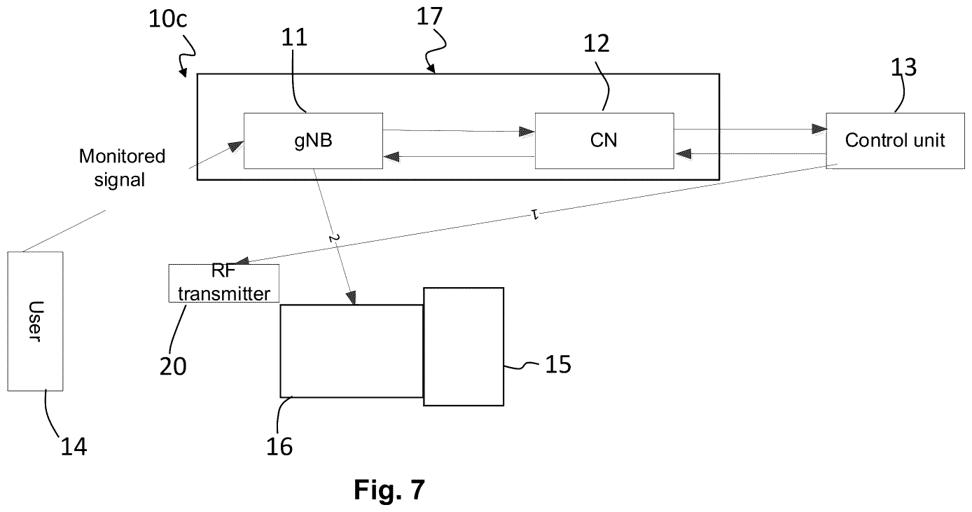

In some embodiments, the first communication to initiate charging of the ambient-powered device 16 may not be transmitted via the mobile telecommunications network 17. For example, FIG. 7 shows a block diagram of a system 10c according to this disclosure. The system 10c of FIG. 7 comprises a local charging transmitter 20 which communicates directly with the control unit 13 in order to receive the first communication indicating that the ambient-powered device 16 is to be charged. The first communication is configured to cause the local charging transmitter 20 to transmit a radiative electromagnetic charging signal to the ambient-powered device 16 in a manner similar to the embodiments discussed above. Once charged, the ambient-powered device 16 may transmit a message via the mobile telecommunications network 17 to the control unit 13 as described above. As such, similar to the other embodiments, the ambient-powered device is configured to communicate, and receive messages from, the mobile telecommunications network 17. The mobile telecommunications network 17 is then configured to transmit the second communication indicating that the ambient-powered device 16 should perform the desired operation.

Thus, in accordance with the above-described embodiments, it will be appreciated that a system 10, 10a, 10b, 10c may be provided to control an ambient-powered device 16 using a mobile telecommunications network 17.

1. A method of controlling an ambient-powered device over a mobile telecommunications network comprising:
receiving, a signal indicating the ambient-powered device is to perform a desired operation;
in response to receiving the signal, transmitting, a first communication to initiate charging of the ambient-powered device; and
transmitting, over the mobile telecommunications network, a second communication to cause the ambient-powered device to perform the desired operation.

2. The method according to claim 1, wherein
the signal is received by the mobile telecommunications network; and
in response to receiving the signal, the mobile telecommunications network transmits the first communication.

3. The method according to claim 1, further comprising after transmitting the first communication, waiting to receive a message from the ambient-powered device indicating the ambient-powered device is charged before transmitting the second communication.

4. The method according to claim 1, wherein the first communication transmitted is a radiative electromagnetic charging signal which charges the ambient-powered device.

5. The method according to claim 1, wherein transmitting the first communication to initiate charging of the ambient-powered device comprises transmitting a first communication over the mobile telecommunications network to a local charging transmitter associated with the ambient-powered electronic device, and
wherein the first communication is configured to cause the local charging transmitter to transmit a radiative electromagnetic charging signal to the ambient-powered device.

6. The method according to claim 5, further comprising:
receiving, over the mobile telecommunications network, location information of the ambient-powered device, wherein
a plurality of local charging transmitters are provided, each local charging transmitter having an associated location, wherein one of the local charging transmitters is associated with the ambient-powered electronic device based on location information of the ambient-powered device.

7. The method according to claim 6, wherein the location information is received from a user terminal associated with the ambient-powered device.

8. The method according to claim 1, wherein
the signal is a signal indicative of a medical treatment to be administered by a user; and
the ambient-powered device is associated with the user and with the medical treatment.

9. The method according to claim 8, wherein the operation performed by the ambient-powered device comprises outputting an alert.

10. The method according to claim 9, wherein the signal indicative of a medical treatment or medical process is received from a control unit for determining a medical treatment or medical process to be administered by the user.

11. A method of operating an ambient-powered device with a mobile telecommunications network comprising:
receiving, by the ambient-powered device, a radiative electromagnetic charging signal to cause the ambient-powered device to charge;
receiving, by the ambient-powered device, a communication from the mobile telecommunications network indicative of an operation to be performed by the ambient-powered device; and
in response to the communication, the ambient-powered device performing the operation.

12. The method according to claim 11, wherein the radiative electromagnetic charging signal is received from the mobile telecommunications network or from a local charging transmitter.

13. The method according to claim 11, wherein the operation to be performed comprises outputting an alert, preferably a sound and/or a light.

14. The method according to claim 11, wherein the radiative electromagnetic charging signal charges a capacitor of the ambient-powered device until the ambient-powered device has stored a predetermined amount of charge, wherein optionally upon storing the predetermined amount of charge, the ambient-powered device transmits a message to the mobile telecommunications network.

15. A mobile telecommunications network configured to perform the method of claim 1.

16. An ambient-powered device configured to perform the method of claim 11.
